(19) United States
(12) Patent Application Publication
(10) Pub. No.: US 2008/0317792 A1
(43) Pub. Date: Dec. 25, 2008

(54) Composition Based on Mineral Concentrates Derived From Precious Stones

(76) Inventors: Jean-Claude Allart, Le Touquet (FR); Jean-Marie Lefevre, Amiens (FR); Jacques Peyrot, Clermont-Ferrand (FR); Jean-Paul Marty, Les Andrets de l'Esterel (FR)

Correspondence Address: CROWELL & MORING LLP;INTELLECTUAL PROPERTY GROUP, P.O. BOX 14300, WASHINGTON, DC, 20044-4300, US

(21) Appl. No.: 12/067,574

(22) PCT Filed: Sep. 22, 2006
(86) PCT No.: PCT/FR06/02174

§ 371 Date: Mar. 20, 2008

(30) Foreign Application Priority Data
Sep. 22, 2005 (FR) 0509706

(51) Int. Cl. A61K 31/74; A61K 8/02; A61K 31/785; A61Q 5/00; A61K 31/715

(52) U.S. Cl. 424/401; 424/70.1; 424/70.16; 424/70.13; 424/70.17

(57) ABSTRACT

The invention relates to a composition based on mineral concentrates.

The composition comprises a powder or a mixture of powders containing, on the one hand, at least one silicon derivative and, on the other hand, at least one trace element other than silicon, as a mixture with a carrier and physiologically acceptable excipients.

Application in cosmetology and dermatology for protecting the skin.

The present invention relates to a novel composition that can be used in cosmetology and in dermatology, and more particularly a novel composition based on mineral concentrates derived from precious stones.

### BACKGROUND OF THE INVENTION

The skin constitutes an organ that is essential to life, which fulfills several functions, the quality of which has a determining influence on the health of any individual. More particularly, the skin constitutes an actual barrier towards the environment and it is subjected to incessant attack. The quality of its defenses is therefore a major component for the effectiveness of its protection.

The skin comprises several integrated layers, namely the surface layer (epidermis) and the deeper layers (the dermis and the hypodermis), and each of these layers has specific properties enabling the whole to react and to adapt to the conditions of its environment.

The epidermis is mainly composed of keratinocytes (90% of the epidermal cells), melanocytes (2 to 3% of the epidermal cells) and Langerhans cells. Its thickness varies over various parts of the body.

The dermis is thicker, and is mainly composed of collagen, elastin and proteoglycans. These three types of molecules are synthesized by the dermal fibroblasts. Collagen fibers provide the mechanical strength and texture of the skin, elastin is responsible for the elasticity, and proteoglycans play a major role in the structure and hydration of the skin. Other cells such as macrophages and leukocytes are also present in the dermis layer.

The hypodermis is the deepest layer of the skin, and contains adipocytes which produce lipids so that the subcutaneous tissue can produce a fatty layer that protects the muscles, the bones and the internal organs against shocks.

Skin aging may be intrinsic, or extrinsic that is to say caused by the environment, including climatic attacks, which may especially contribute to accelerating the degradation of the collagen of the dermis, and in particular sun exposure, temperature variations and free radicals. The first signs of skin aging, such as wrinkles and fine lines, are generally caused by stress and biological and physiological changes, accelerated by the outside environment or by lifestyle. The appearance of pigment marks, the decrease in the thickness of the skin and its sagging are also changes observed during aging. It is known that the ability of the skin to replace damaged collagen decreases over time, and consequently gaps and irregularities appear in the collagen network.

Thus, the skin functions are constantly stressed, and especially the epidermis through its keratinization function, the dermis through its fibroblast function, the dermal-epidermal junction through its anchoring function and its regulating function, and also microcirculation through its nutritional and oxygenating function.

Skin protection must focus, as a priority, on the cutaneous surface as any disturbance of the stratum corneum and of the surface hydrolipid film has effects on the deeper structures of the skin. Vascularization and microcirculation must also be taken into consideration in order to ensure the maintenance of a good cutaneous equilibrium.

Numerous compositions intended to protect the skin against the damaging effects of ultraviolet radiation are known, and these compositions may contain screening agents or pigments that form a sunscreen. For example, it is possible to use hydrophilic or lipophilic UV-A and UV-B sunscreens which may be chosen from benzophenone or a benzophenone derivative such as 2-hydroxy-4-methoxybenzophenone (EUSOLEX® 4360), or a cinnamic acid ester such as 2-ethylhexyl methoxycinnamate (PARSOL MCX®), or else a cyano-β,β-diphenylacrylate such as octocrylene (EUSOLEX® OCR), 4-methylbenzylidene camphor (EUSOLEX 6300®), and derivatives of dibenzoylmethane such as 4-isopropyl dibenzoylmethane (EUSOLEX 8020), and 4-methoxydibenzoylmethane. These screening agents may be used separately or in combination, as in patents EP 514 491 and EP 685 225. It is also possible to use pigments that form an ultraviolet-resistant screen; these pigments may, for example, be chosen from titanium dioxide, zinc oxide, zirconium oxide or else aluminum oxide. In particular, it is possible to use metal oxide nanopigments having a particle size between 5 and 100 nm, such as those described in Patent Application EP 518 773.

Various treatments have also been proposed for protecting skin and reducing the signs of skin aging. For example, treatments based on compositions such as creams and lotions that contain α-hydroxy acids or retinoids, applied regularly, have been proposed for gradually reducing the number of wrinkles and fine lines.

However, although all these known products and methods may have a favorable effect on the signs of skin aging, for example by protecting the skin, or by masking or reducing wrinkles, they generally only have a limited effect on the development which leads to these signs of aging.

Furthermore, some cosmetic compositions are known that comprise mineral compounds. For example, U.S. Pat. No. 4,857,306 describes compositions intended for makeup which may contain powders based on colored precious stones in a highly viscous liquid carrier. However, these various mineral compounds derived from precious stones have been used in compositions due to their colored or particle size properties; on the other hand, no skin protection property has been demonstrated. Application WO 2005/044185 describes a composition intended for protecting the skin against electromagnetic emissions that contains a combination of a cationic compound, a mineral compound such as fuchsite or malachite and metal particles. Patent FR 2805743 describes the use of nephrite jade powder in a composition due to its known medical effects. U.S. Pat. No. 5,985,021 describes the use of silimanite (alumina silicate), topaz, zeolite and hematite in soaps and cosmetic compositions due to the beneficial effects that they are supposed to have on the skin. Patent Application US 2002/012681 relates to a cosmetic composition comprising mineral powders having fluorescence properties. However, these compositions do not make it possible to procure both an effective protection against radiation and an activation of the microcirculatory functions of the skin.

It therefore appeared desirable to develop cosmetic or dermatological compositions capable of providing an excellent surface protection for the skin and more particularly a composition providing:

protection against the damaging effects of electromagnetic radiation and against the physical and chemical attacks of the environment in order to preserve its natural defenses, to maintain a good local hydration and to preserve its fibroblast function; and

activation of the microcirculatory functions in order to improve the nutrition, drainage and oxygenation of the skin.

### SUMMARY OF THE INVENTION

The subject of the present invention is therefore a composition that can be used in cosmetology and in dermatology providing skin protection, especially against electromagnetic radiation and the attacks of the environment, favoring skin microcirculation and capable of maintaining its hydration.

The composition according to the present invention, that can be used in cosmetics and in dermatology to provide skin protection as indicated above, is differentiated in that it comprises, on the one hand, at least one silicon derivative and, on the other hand, at least one other trace element, as a mixture with a carrier and physiologically acceptable excipients.

### BRIEF DESCRIPTION OF THE DRAWINGS

FIG. 1 shows the absorption spectrum in a wavelength range from 190 nm to 900 nm indicated on the x-axis.

FIG. 2 shows the reflectance spectrum of the mineral concentrate B described in Example 1 below in comparison with titanium oxide (TiO₂) taken as a reference.

FIG. 3 shows the reflectance spectrum of the composition corresponding to the mineral concentrate F described in Example 1, titanium oxide being taken as a reference.

### DETAILED DESCRIPTION OF THE PREFERRED EMBODIMENTS

More particularly, the composition comprises, on the one hand, a dry powder or a mixture of dry powders obtained from precious or semiprecious stones containing at least one silicon derivative and, on the other hand, an aqueous suspension of powders obtained from precious or semiprecious stones containing at least one trace element other than silicon, to form a mineral concentrate, as a mixture with a carrier and physiologically acceptable excipients.

The composition according to the present invention preferably comprises a powder or a mixture of powders containing, on the one hand, a silicon oxide (silica) and/or a silicate and, on the other hand, one or more trace elements, other than silicon.

The trace elements are chosen from iron, copper, zinc, manganese, molybdenum, boron, cobalt, aluminum and selenium.

These powders and mixtures of powders based on a derivative of silicon and/or silicate, and trace elements, are preferably obtained from precious or semiprecious stones and more particularly tourmaline, aquamarine, emerald, citrine, amethyst, malachite, rhodochrosite, smithsonite, rhodolite, lapis lazuli, sapphire, ruby and topaz.

Preferably, precious or semiprecious stones are chosen that contain silicon or silicates, and for example tourmaline which contains borosilicate, aquamarine and emerald which contain an aluminosilicate, and citrine and amethyst which contain silicon dioxide. The precious or semiprecious stones containing trace elements are preferably chosen from malachite which contains copper, rhodochrosite which contains manganese, sapphire which contains aluminum and smithsonite which contains zinc.

Thus, according to one preferred embodiment of the invention, the composition contains, on the one hand, at least one powder obtained from precious or semiprecious stones containing a silicon derivative chosen from a borosilicate, an aluminosilicate and silicon dioxide and, on the other hand, at least one powder containing a trace element chosen from iron, copper, zinc and manganese. The precious or semiprecious stones containing a silicon derivative are preferably chosen from aquamarine, tourmaline, emerald, citrine and amethyst, whereas the precious stones containing a trace element are preferably chosen from malachite, rhodochrosite, sapphire and smithsonite.

These precious or semiprecious stones are available in powder form and are reduced to powder by milling so as to obtain a particle size between 1 μm and 50 μm. The average size of the powder particles is preferably less than 50 μm, and more particularly less than around 20 μm.

For example, aquamarine is an ultrafine powder, having an average particle size of less than 10 microns. Its general formula comprises an aluminosilicate of beryllium with chromium and traces of magnesium. Its crystal system is hexagonal with a column arrangement. The heavy metal doping is less than 50 ppm.

Citrine is an ultrafine powder of pale gray color with an average particle size of less than 10 microns. Its general formula comprises silicon dioxide of iron and manganese, calcium and titanium. Its crystal system is hexagonal with six sides. Its content of heavy metals is less than 50 ppm.

According to one preferred embodiment, the composition comprises, as a mixture, one or more dry powders containing the silicon derivative and an aqueous suspension comprising one or more powders containing the trace elements, to form a mineral concentrate.

The dry powders/suspension of powders weight ratio may be between 1/5 and around 1/1, this ratio not being limiting.

Thus, according to one preferred embodiment of the invention, the composition comprises a mixture of dry powders obtained by milling aquamarine, tourmaline, citrine and amethyst, and an aqueous suspension of powders obtained by milling malachite, rhodochrosite and smithsonite, and also a carrier and physiologically acceptable excipients.

The expression “physiologically acceptable”, within the meaning of the present invention, is understood to mean carriers and excipients of a type commonly used in cosmetic and dermatological compositions, that are neutral with respect to the active principles used, that do not have a toxic effect and do not give rise to any secondary effect that is damaging to the skin.

According to one variant, it is advantageous to add sapphire in powder from, in an amount representing between 1 and 20 wt % of the composition, and preferably between 5 and 15 wt %, to the above composition. The average size of the particles may be between 5 and 20 μm. Added in powder form, sapphire substantially improves the ultraviolet radiation reflectivity and thus increases the solar protection power of the compositions according to the invention.

The powders containing trace elements such as iron, zinc, copper and manganese may generally be used in the form of commercially available aqueous extracts or suspensions. These aqueous extracts or suspensions are soluble in water and are generally insoluble in mineral and plant oils. Their solids content is generally between 0.2% and 10% and their trace element content between 1 and 15%, or even higher. The usage concentration of these aqueous extracts or suspensions may be of the order of 0.5% to 50% and preferably from 1% to 30%.

It may be advantageous to incorporate into the composition a substance that improves the coverage, such as, for example, boron nitride. This substance is added after mixing the dry powders and the suspended powders.

A binder such as a polyol, polyethylene glycol, butylene glycol and glycerol may advantageously be incorporated into the composition of the mineral concentrate. According to the invention, preferably glycerol is used.

A gelling agent is preferably added to improve the physical properties of the composition. It is more particularly possible to use a gelling agent chosen from polyacrylamides (for example, of the Carbopol type), acrylate/acrylic acid or acrylamide/acrylamidopropanesulfonic acid copolymers, mixtures based on polyacrylamide, and for example a mixture of polyacrylamide, C13-14 isoparaffin and Laureth-7 (SEPIGEL 305® from Seppic), cellulose derivatives such as hydroxypropyl cellulose, chitosan, plant mucopolysaccharides and clays.

The mineral concentrate containing, where appropriate, boron nitride is diluted in the gelling agent, and the excipients are added so that the content of the mineral concentrate in the final composition is between 1% and 15%, preferably between 2% and 10% by weight relative to the total weight of the composition.

Tests carried out with various mineral concentrates according to the invention have demonstrated advantageous properties that can be used in cosmetology and in dermatology, especially direct skin protection effects, in particular a solar protection effect, an antioxidant effect and a thermal and microcirculatory effect.

### Solar Protection

Tests were carried out with mineral concentrates diluted to 2.5%, 5% and 10% respectively, in a neutral carrier which may contain the gelling agent (SEPIGEL 305) optionally with the addition of binder, to evaluate the ability to protect against radiation, by comparison with a known sunscreen based on titanium oxide, and they demonstrated an excellent efficiency in a wavelength range of 190 to 900 nm, that is to say an ultraviolet to infrared range.

It is particularly interesting to observe that the results are substantially equivalent to the three concentrations tested of 2.5%, 5% and 10%, which shows that a satisfactory protective effect against radiation is obtained from the dose of 2.5% and that, beyond this value, the effect no longer depends on the dose.

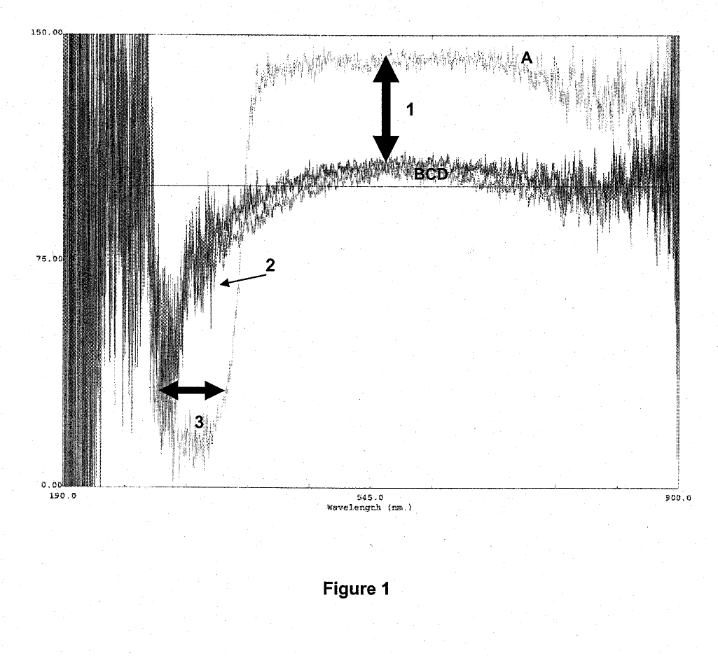

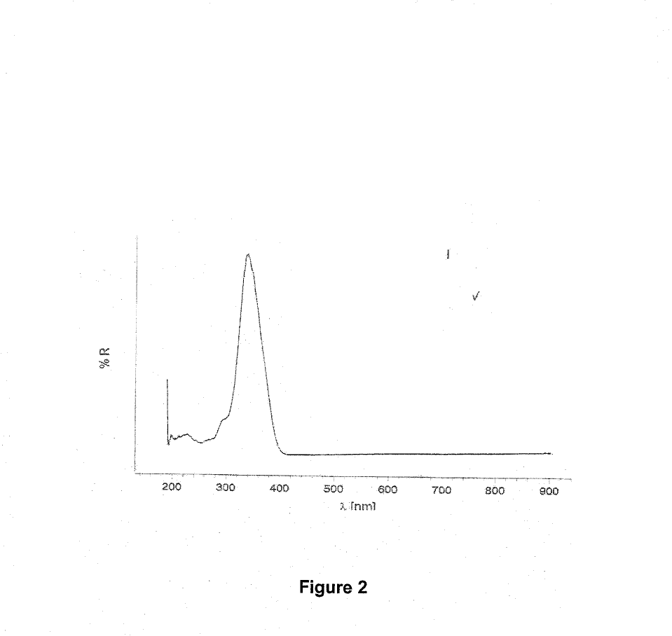

More particularly, by using the mineral concentrate having the composition A indicated in Example 1, the absorption and reflection of UV to IR radiation were verified by spectroscopy in comparison with titanium oxide. The spectra are represented in the appended FIGS. 1 and 2.

FIG. 1 shows the absorption spectrum in a wavelength range from 190 nm to 900 nm indicated on the x-axis. The curve A corresponds to titanium oxide (TiO₂) taken as a reference, whereas the curves B, C and D correspond to the mineral concentrate A of Example 1, at the concentrations of 10%, 5% and 2.5% respectively. These three curves are practically superposed, which shows that the absorption effect does not depend on the dose beyond 2.5%. The gap (1) between curve A and curves B, C and D shows that the mineral concentrate of the invention has a higher absorption than titanium oxide. The section (2) of the curves shows that the mineral concentrate has an absorption equivalent to that of titanium oxide in the ultraviolet range, and the gap (3) shows that the absorption of titanium is greater in this zone.

FIG. 2 shows the reflectance spectrum of the mineral concentrate B described in Example 1 below in comparison with titanium oxide (TiO₂) taken as a reference. This spectrum shows that in the ultraviolet zone, the mineral concentrate of the invention has a reflectance value greater than that of titanium oxide.

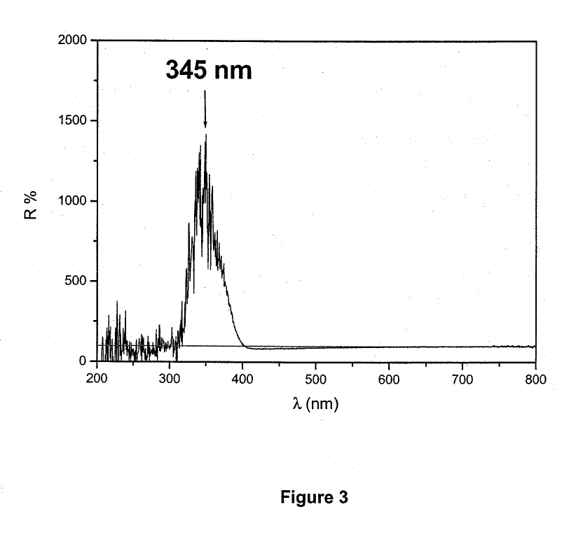

FIG. 3 shows the reflectance spectrum of the composition corresponding to the mineral concentrate F described in Example 1, titanium oxide being taken as a reference. This spectrum clearly shows a very marked increase in the reflectivity in the ultraviolet range centered around 345 nm.

These results show that the compositions according to the invention provide an effective protection against radiation, in particular UV rays, without it being necessary to add a UV screening agent or a sunscreen.

### Direct Protection

The immediate protective effect has been verified by a stinging test. This test consists in producing an irritation by applying a drop of 10% lactic acid onto one of the nasal folds of a patient, then in comparing with a placebo on the other nasal fold. The placebo used is physiological serum. The composition of the invention was first applied to the skin, before application of the lactic acid and the physiological serum, on half of the patients. The composition of the invention is the mineral concentrate A from Example 1 diluted to 2.5% in SEPIGEL 305. The test is carried out on a sample of 10 patients having delicate and sensitive skin.

The degree of irritation is evaluated on a scale of 0 (no irritation) to 10 (significant irritation). The average for the patients who have not received the composition of the invention is 7.5 whereas it is only 3.5 in patients who have received it.

This test shows the protective effect of the mineral concentrate according to the present invention.

### Antioxidant Effect

The mineral concentrate according to the invention has shown an unexpected antioxidant effect measured by in vivo tests.

The evaluation of the antioxidant effect was carried out by measuring the degradation of squalene under ultraviolet radiation. Squalene is a natural compound present in skin sebum which, due to its unsaturated chemical structure, is easily oxidized after exposure to ultraviolet rays. The oxidation product may be assayed by HPLC chromatography. The principle of the test therefore consists in measuring the intensity of the characteristic HPLC peak of the squalene and of its oxidation product (monohydroperoxide), after controlled exposure to ultraviolet rays.

The evaluation procedure consists in applying, to the skin, the mineral concentrates A and B of the invention described in Example 1, then in removing a sample of sebum from the forehead of each volunteer by means of a strip of SEBUTAPE® after 1 hour of contact with the product, then irradiation with ultraviolet rays (Philips UVA-B lamp, delivering 30-40 joules/cm², placed at 21 cm for 16 minutes) in order to oxidize the squalene present in the removed sebum.

The surface of the skin was cleaned 5 minutes before the start of the test using a swab soaked with 70° ethyl alcohol so as to remove the lipids present at the surface. Two sebum samples were removed from each volunteer participating in the test by means of two strips of SEBUTAPE® and each strip was divided into two parts, of which one was exposed to UV rays and the other was not in order to act as a reference. The same tests were carried out with a placebo (same composition without the mineral concentrate of the invention).

The average values of the amounts of non-oxidized squalene detected in the sebum for the mineral concentrates A and B according to the invention, at T0 (immediately after application of the composition) and at T1 (1 hour after application), are indicated in the following table (amounts in μg in the SEBUTAPE® strip)

| | T0 | T1 |
|---|---|---|
| Unirradiated mineral | 54.45 ± 48.93 | 56.67 ± 21.34 |
| concentrate A | | |
| Irradiated mineral concentrate | 10.68 ± 10.53 | 14.56 ± 7.61 |
| A | | |
| Unirradiated mineral | 45.64 ± 26.11 | 64.48 ± 28.64 |
| concentrate B | | |
| Irradiated mineral concentrate | 11.04 ± 9.69 | 21.17 ± 11.37 |
| B | | |

By being based on a value of 100 for the unirradiated sample, the relative amounts of non-oxidized squalene are expressed in the table below:

| | T0 | T1 |
|---|---|---|
| Mineral concentrate A | 19% | 26% |
| Mineral concentrate B | 26% | 33% |

These results show the effectiveness of the mineral concentrate of the invention against the oxidation of squalene by exposure to ultraviolet rays. The application of a composition according to the invention to the skin before exposure to the ultraviolet rays induces a sebum protective effect.

Furthermore, the tests show that the composition of the invention does not have an oxidizing effect, even after exposure, unlike certain oils whose degradation products are oxidizing and have a harmful effect for the skin, in particular monoi oils or certain benzophenones used as solar protection products. This result is all the more advantageous since the composition of the invention provides protection against ultraviolet radiation, as indicated above, which then renders the addition of UV screening agents less useful.

Moreover, it has been observed that the mineral concentrate of the invention does not modify sebum excretion and therefore does not have an occlusive effect, and that it may even have a sebum-absorbing effect.

### Effect on Cutaneous Microcirculation

The study was carried out on a sample of 5 women aged between 23 and 32 years old.

The averages of the results are given in the table below. A first measurement is carried out at the time of application of the mineral concentrate A to the skin (T0) and a second measurement is carried out 5 minutes later (T1). A single application of 50 mg of mineral concentrate is made to the skin of the cheek and the cheekbone on one side of the face. Measurements of skin thermography are made by means of a camera placed at 62 cm from the patient's face. Measurement of the circulatory flow is carried out in darkness using a Doppler laser.

| Average values | T0 | T1 |
|---|---|---|
| Thermograph | 29.65 | 29.26 |
| Laser (flow) | 396.16 | 397.52 |
| Thermocouple | 0.65 | 0.45 |

This study shows that the application of the mineral concentrate A described in Example 1, to the skin, leads to thermal modifications observed by thermography and by thermocouple. These modifications are observed only 5 minutes after application to the skin.

The thermal reduction observed (thermograph and thermocouple) proves that the mineral concentrate of the invention very rapidly exerts a shielding effect by decreasing the heat losses at the surface of the skin, without reducing the circulatory flow measured by the Doppler laser. This result is unexpected as a reduction in the circulatory flow would have been anticipated according to the conventional rules of thermoregulation linked to the microcirculation. On the contrary, the slight increase in the flow observed is expressed by an energetic action on the vasomotricity induced by the mineral concentrate of the invention.

These various tests show that the mineral concentrate according to the invention provides a protective effect against radiation over a range of wavelengths from the ultraviolet to the infrared, against skin irritation, antioxidant protection, with a thermal surface effect, stimulation of the microcirculation, a sebum-absorbing ability without occlusive effect, and also perfect tolerance of the skin towards the mineral concentrate without a sensitization phenomenon.

This mineral concentrate constitutes an active principle having useful properties that make it possible to incorporate it into topical compositions for dermatological or cosmetological use, in a concentration which may be between 1 and 50 wt % relative to the total weight of the composition, preferably between 1 and 20 wt % and more preferably between 2 and 10 wt % for the purpose of protecting the skin or strengthening its natural defenses.

According to one embodiment of the present invention, it may be advantageous to incorporate, into the composition, a substance that provides a complementary activity that can be used, for example, to promote collagen synthesis and, more particularly, a lipopeptide such as palmitoyl-lysyl-threonyl-threonyl-lysyl-serine (MATRIXYL® from Sederma), generally in the form of an aqueous-alcoholic solution.

The mineral concentrate may also be used to reinforce the action of other active agents present in such topical compositions to promote skin hydration, solar protection, the fight against inflammatory phenomena, the fight against skin aging and more generally the maintenance of good homeostasis.

The moisturizer or humectant optionally incorporated into the composition may be chosen from moisturizers conventionally used in cosmetic or dermatological compositions, for example a polyol, glycerine (glycerol and glycerol derivatives), diglycerine, polyethylene glycol, sorbitol, glyceryl polyacrylates and polymethacrylates, mucopolysaccharides such as hyaluronic acid, chitosan derivatives and derivatives of pyrrolidone carboxylic acid. The moisturizer or humectant content is generally between 0.1 and 10% by weight relative to the total weight of the composition.

The customary preservatives of the art of dermatological or cosmetological compositions may be used in the invention, for example an alcohol such as ethanol, isopropanol and phenoxyethanol, benzoic acid and an alkyl p-hydroxybenzoate such as methyl and propyl p-hydroxybenzoates (methylparaben and propylparaben), or else chlorophenesin or imidazolidinyl urea. These preservatives may be used separately or in combination.

The composition may be completed by various excipients depending on the nature of the desired phases, such as C8-C10 caprylic/capric triglyceride (ESTASAN or MIGLYOL 812), or oleic acid, as constituents of the fatty phase in the case of an emulsion, or cyclopentasiloxane to improve the feel properties of the composition.

The compositions according to the invention may be in the form of creams, balms, oil-in-water (O/W) emulsions or water-in-oil (W/O) emulsions, gels, serums, masks, ointments or pomades, and preferably in the form of creams or water-in-silicone or oil-in-water emulsions. These various galenic forms are prepared according to the customary techniques. These compositions may be presented in the form of a makeup supplement or care products, for example lip balm.

The following examples illustrate the invention in greater detail without limiting the scope thereof. In these composition examples, the parts are expressed by weight, except where mentioned otherwise.

### EXAMPLE 1

### Preparation of a Mineral Concentrate

Poured successively into a 1 l container equipped with a mixer were the following powders:

| | |
|---|---|
| aquamarine (average size 10 μm) | 2.5 g |
| tourmaline (average size 10 μm) | 15.0 g |
| citrine (average size 10 μm) | 7.5 g |

After having carefully mixed the powders for around 15 minutes, the aqueous extracts indicated below were introduced:

| | |
|---|---|
| MALAKITE ® (aqueous extract having 5.0 g/l of copper) | 15.0 g |
| RHODOLITE ® (aqueous extract having 3.0 g/l of manganese) | 30.0 g |

MALAKITE® was an aqueous extract of malachite available commercially (Gattefossé) having a copper content greater than 5 g/l and of which the solids content was between 5 and 7%. RHODOLITE® was an aqueous extract of rhodochrosite available commercially (Gattefossé) having a manganese content between 1.0 and 3.5 g/l and of which the solids content was between 0.7 and 1.5%.

The mixture obtained continued to be stirred at ambient temperature for around 15 minutes, in order to form a mineral concentrate, then a gelling agent was added, composed of 2.0 g of a mixture of polyacrylamide, C13-14 isoparaffin and Laureth-7 (SEPIGEL 305®) with the addition of 16.5 g of boron nitride (white powder with an average grain size equal to 2 μm).

Thus, a mineral concentrate A was obtained to which a gelling agent and boron nitride had been added, having the weight composition below expressed in parts by weight:

### Mineral Concentrate A

| | |
|---|---|
| aquamarine | 2.5 |
| tourmaline | 15.0 |
| citrine | 7.5 |
| MALAKITE ® | 15.0 |
| RHODOLITE ® | 30.0 |
| glycerine | 11.5 |
| gelling agent | 2.0 |
| boron nitride | 16.5 |

The gelling agent used was SEPIGEL 305 as for the preceding mineral concentrate.

### Mineral Concentrate B

By proceeding in the same manner, a mineral concentrate B was prepared to which a gelling agent and boron nitride were added, having the composition below expressed in parts by weight:

| | |
|---|---|
| aquamarine | 2.5 |
| tourmaline | 20.0 |
| citrine | 10.0 |
| MALAKITE ® | 7.5 |
| RHODOLITE ® | 15.0 |
| glycerine | 20.0 |
| boron nitride | 25.0 |

The glycerine was used to absorb water and thicken the composition.

### Mineral Concentrate C

By proceeding in the same manner as above, the following mineral concentrate was prepared:

| | |
|---|---|
| aquamarine | 7.0 |
| tourmaline | 15.0 |
| amethyst | 3.0 |
| MALAKITE ® | 25.0 |
| RHODOLITE ® | 25.0 |
| glycerine | 8.0 |
| gelling agent | 2.0 |
| boron nitride | 15.0 |

### Mineral Concentrate D

| | |
|---|---|
| aquamarine | 5.0 |
| tourmaline | 15.0 |
| citrine | 5.0 |
| MALAKITE ® | 22.5 |
| RHODOLITE ® | 22.5 |
| glycerine | 11.0 |
| gelling agent | 2.5 |
| boron nitride | 16.5 |

### Mineral Concentrate E

| | |
|---|---|
| aquamarine | 5.0 |
| tourmaline | 15.0 |
| citrine | 5.0 |
| MALAKITE ® | 22.5 |
| ZIN'CITE ® | 22.5 |
| glycerine | 11.0 |
| gelling agent | 2.5 |
| boron nitride | 16.5 |

ZIN'CITE® was an aqueous extract of smithsonite available commercially (Gattefossé) having a zinc content between 1.2 and 2.5 g/l and of which the solids content was between 0.4 and 1.0%.

### Mineral Concentrate F

| | |
|---|---|
| tourmaline | 25.0 |
| citrine | 0.5 |
| MALAKITE ® | 5.0 |
| sapphire | 10.0 |
| glycerine | 30.0 |
| boron nitride | 25.0 |
| water | 4.5 |

### EXAMPLE 2

Via the customary techniques, a day cream was prepared having the following composition indicated in parts by weight:

| | |
|---|---|
| mineral concentrate A above | 5.00 |
| EDTA | 0.05 |
| PEG 400 | 3.00 |
| glycerine | 4.00 |
| phenoxyethanol | 0.80 |
| chlorophenesin | 0.10 |
| caprylic/capric triglyceride | 6.00 |
| polyisobutene | 4.00 |
| CETYL ALCOHOL (and) GLYCERYL STEARATE (and) | 4.00 |
| PEG-75 STEARATE (and) CETETH-20 (and) | |
| STEARETH-20 | |
| Cera alba | 0.50 |
| macadamia nut oil | 1.00 |
| octenyl succinate | 1.00 |
| lipoprotein complex | 2.00 |
| cyclopentasiloxane | 3.00 |
| sodium acrylate | 2.00 |
| water | qs for 100.00 |

This cream was intended for use by applying to the skin of the face and of the décolletage one to two times per day, for 6 to 12 weeks. It provides good hydration and excellent protection against attacks of the environment, in particular against ultraviolet rays.

### EXAMPLE 3

Via the customary techniques, a day cream was prepared having the following composition indicated in parts by weight:

| | |
|---|---|
| mineral concentrate B above | 5.00 |
| propylene glycol | 5.00 |
| octyl palmitate | 2.00 |
| polyisobutene | 7.00 |
| phenoxyethanol | 0.80 |
| chlorophenesin | 0.20 |
| caprylic/capric triglyceride | 6.00 |
| dimethicone | 0.20 |
| acrylate/C10-C30 alkylacrylate crosspolymer | 0.30 |
| beeswax | 0.50 |
| tocopheryl acetate | 0.10 |
| carbomer | 0.50 |
| triethanolamine | 0.60 |
| MATRIXYL ® | 2.00 |
| water | qs for 100.00 |

This cream could be used by applying to the skin of the face one or two times per day, for 10 weeks and provides good hydration and excellent protection against the attacks of the environment, in particular against ultraviolet rays.

### EXAMPLE 4

By the customary techniques, a makeup-removing milk was prepared having the following composition indicated in parts by weight:

| | |
|---|---|
| mineral concentrate A above | 5.00 |
| glyceryl stearate | 4.50 |
| stearic acid | 3.00 |
| cetyl palmitate | 0.30 |
| polyisobutene | 7.00 |
| sorbic acid | 0.10 |
| chlorophenesin | 0.20 |
| caprylic/capric triglyceride | 3.00 |
| monopropylene glycol | 3.00 |
| allantoin | 0.30 |
| shea butter | 0.80 |
| tocopheryl acetate | 0.10 |
| sodium alginate | 0.30 |
| phenoxyethanol | 0.80 |
| triethanolamine | 0.60 |
| MATRIXYL ® | 2.00 |
| water | qs for 100.00 |

This milk was used once a day for removing makeup from the skin.

### EXAMPLE 5

A corrector serum was prepared by the customary manufacturing techniques, that had the following weight composition:

| | |
|---|---|
| mineral concentrate C above | 10.00 |
| chlorophenesin | 0.20 |
| CARBOMER ® | 0.45 |
| glycerine | 10.00 |
| triethanolamine | 0.80 |
| xanthan gum | 0.20 |
| MATRIXYL ® | 2.00 |
| water | qs for 100.00 |

This serum was applied to the skin in the evening by massaging into the face and décolletage until it had completely penetrated, and it provides a tightening effect of the skin.

### EXAMPLE 6

A treating serum was prepared that contained the mineral concentrate F described in Example 1 and that had the following composition, according to a customary method described below.

| | |
|---|---|
| POLYSORBATE 20 ® | 2.00 |
| alkylate/C₁₀₋₃₀ alkylacrylate crosspolymer (ULTREZ 21 ®) | 0.30 |
| glycerine | 5.00 |
| diglycerine | 4.00 |
| disodium EDTA | 0.10 |
| preservative | 1.00 |
| polyvinyl alcohol | 0.20 |
| water | 20.00 |
| SIMULGEL NS ® | 1.00 |
| isodecyl neopentanoate | 3.00 |
| cetearyl ethylhexanoate | 1.00 |
| Mineral concentrate F above | 1.00 |
| PLASTIC POWDER D 400 ® | 3.00 |
| Methyl methacrylate crosspolymer (MICROPEARL M310 ®) | 3.00 |
| cyclopentasiloxane | 7.00 |
| ethyl alcohol 96° | 3.00 |
| MATRIXYL 3000 ® | 7.00 |
| fragrance | 0.50 |

The emulsifier (POLYSORBATE 20), the gelling agent (ULTREZ 21), the moisturizer (glycerine, diglycerine), the EDTA, the polyvinyl alcohol, the preservatives (for example chlorophenesin and phenoxyethanol) and the water were mixed at high temperature (40-45° C.) and added to the mixture were SIMULGEL, isodecyl neopentanoate and cetearyl ethylhexanoate, and optionally 0.20 parts of 40% sodium hydroxide until a homogenous mixture was obtained. After cooling to 30-35° C., with stirring, the mineral concentrate F described in Example 1, then the other ingredients, were added. The MATRIXYL and the fragrances were added at ambient temperature.

1. A cosmetic and/or dermatological composition for protecting the skin comprising a dry powder or a mixture of dry powders obtained from precious or semiprecious stones containing at least one silicon derivative and an aqueous suspension of powders obtained from precious or semiprecious stones containing at least one trace element other than silicon, to form a mineral concentrate, as a mixture with a carrier and physiologically acceptable excipients.

2. The composition as claimed in claim 1, wherein it comprises a powder or a mixture of powders containing a silicon oxide (silica) and/or a silicate and one or more trace elements.

3. The composition as claimed in claim 1, wherein the precious or semiprecious stones are selected from the group consisting of tourmaline, aquamarine, emerald, citrine, amethyst, malachite, rhodochrosite, smithsonite, lapis lazuli, ruby, sapphire and topaz.

4. The composition as claimed in claim 1 wherein, the trace element is selected from the group consisting of iron, copper, zinc, manganese, molybdenum, boron, cobalt, aluminum and selenium.

5. The composition as claimed in claim 1, wherein it comprises at least one powder obtained from precious or semiprecious stones containing a silicon derivative selected from the group consisting of a borosilicate, an aluminosilicate and silicon dioxide.

6. The composition as claimed in claim 5, wherein the precious or semiprecious stones are selected from the group consisting of aquamarine, tourmaline, citrine and amethyst.

7. The composition as claimed in claim 1, wherein it comprises at least one powder obtained from precious or semiprecious stones containing a trace element selected from the group consisting of iron, copper, zinc and manganese.

8. The composition as claimed in claim 7, wherein the precious or semiprecious stones are selected from the group consisting of malachite, rhodochrosite and smithsonite.

9. The composition as claimed in claim 1, wherein the particle size of the powders is between 1 μm and 50 μm.

10. The composition as claimed in claim 1, wherein it comprises a mixture of dry powders obtained by milling aquamarine, tourmaline, emerald, citrine and amethyst, and an aqueous suspension of powders obtained by milling malachite, rhodochrosite and smithsonite, and also a carrier and physiologically acceptable excipients.

11. The composition as claimed in claim 1, wherein it additionally comprises a gelling agent.

12. The composition as claimed in claim 11, wherein the gelling agent is selected from the group consisting of polyacrylamides, acrylate/acrylic acid or acrylamide/acrylamidopropanesulfonic acid copolymers, cellulose derivatives, chitosan, plant mucopolysaccharides and clays.

13. The composition as claimed in claim 12, wherein the gelling agent is hydroxypropyl cellulose or a mixture of polyacrylamide, C13-14 isoparaffin and Laureth-7.

14. The composition as claimed in claim 10, wherein it additionally comprises boron nitride.

15. The composition as claimed in claim 10, wherein it additionally comprises a sapphire powder.
